SEQUENCE LISTING (12 sequences)

SEQ ID NO: 1 - DNA, 20 bp, Artificial
  actin sense
tcagaagaactcctatgtgg

SEQ ID NO: 2 - DNA, 20 bp, Artificial
  actin antisense
tctctttgatgtcacgcacg

SEQ ID NO: 3 - DNA, 26 bp, Artificial
  tnf-alpha sense
cacgctcttctgtctactgaacttcg

SEQ ID NO: 4 - DNA, 26 bp, Artificial
  tnf-alpha antisense
ggctgggtagagaatggatgaacacc

SEQ ID NO: 5 - DNA, 20 bp, Artificial
  IL-1beta sense
ggatgaggacatgagcacct

SEQ ID NO: 6 - DNA, 20 bp, Artificial
  IL-1beta antisense
tccattgaggtggagagctt

SEQ ID NO: 7 - DNA, 24 bp, Artificial
  IL-6 sense
tgaagttcctctctgcaagagact

SEQ ID NO: 8 - DNA, 19 bp, Artificial
  IL-6 antisense
tgaggaaggccgtggttgt

SEQ ID NO: 9 - DNA, 16 bp, Artificial
  UCN sense
caggcgagcggccgcg

SEQ ID NO: 10 - DNA, 19 bp, Artificial
  USN antisense
cttgcccaccgagtcgaat

SEQ ID NO: 11 - DNA, 21 bp, Artificial
  CRH sense
caactttttccgcgtgttgct

SEQ ID NO: 12 - DNA, 21 bp, Artificial
  CRH anti-sense
atggcataagagcagcgctat

(19) United States
(12) Patent Application Publication
(10) Pub. No.: US 2009/0247558 A1
(43) Pub. Date: Oct. 1, 2009

(54) USE OF THE CRH (CORTICOTROPIN RELEASING HORMONE) - UCN (UROCORTIN) SYSTEM IN THE TREATMENT OF INFLAMMATORY DISEASES

(75) Inventors: Andrew N. MARGIORIS, Heraklion (GR); Achille Gravanis, Heraklion (GR)

Correspondence Address: SUGHRUE MION, PLLC, 2100 PENNSYLVANIA AVENUE, N.W., SUITE 800, WASHINGTON, DC, 20037, US

(73) Assignee: BIONATURE E.A. LIMITED, NICOSIA (CY)

(21) Appl. No.: 12/409,300

(22) Filed: Mar. 23, 2009

Related U.S. Application Data
(62) Division of application No. 10/535,307, filed on Dec. 7, 2005, now abandoned.

(30) Foreign Application Priority Data
Nov. 26, 2002 (GR) 20020100513

(51) Int. Cl. A61K 31/519; A61P 29/00

(52) U.S. Cl. 514/265.1

(57) ABSTRACT

The invention relates to the use of corticotropin-releasing hormone (CRH) receptor-1 (R1) antagonists and/or CRH-R2 receptor agonists for the treatment of inflammatory diseases via regulation of monocyte/macrophage cell activation, proliferation, differentiation, apoptosis, and inflammatory cytokine production. As CRH system we define natural and synthetic CRH and urocortin (UCN) agonists and antagonists for the CRH-R1 and CRH-R2 receptors and their subtypes as well as the CRH-binding protein (BP), a CRH pseudo-receptor. The invention is directed towards pharmacological intervention for the amelioration or treatment of inflammatory diseases using the CRH system-mediated control of monocyte/macrophage cells which play a key role in initiating and maintaining the inflammatory response via production of pro-inflammatory cytokines such as is the interleukin (IL)-1, IL-6 and tumor necrosis factor (TNF)-alpha. By the term inflammation we define the response of an organism to noxious endogenous or exogenous stimuli causing tissue injury. Inflammation is a host defence mechanism, which might harm the defending organism. The invention also provides methods for the in vitro and in vivo evaluation of natural and synthetic CRH system modulators for the control of the monocyte/macrophage system.

This is a Divisional Application of U.S. application Ser. No. 10/535,307, filed Dec. 7, 2005, which is a National Stage entry of PCT/IB03/05429, filed Nov. 26, 2003, which claims priority from GR Application No. 2002-0100513, filed Nov. 26, 2002. The Entire disclosures of the prior applications are incorporated herein by reference.

The present invention relates to pharmaceutical compositions for the treatment of inflammatory diseases. In particular the invention relates to pharmaceutical compositions comprising a synthetic CRH-R1 antagonist and/or synthetic CRH-R2 agonist.

In another aspect the invention relates to the treatment of inflammatory diseases using synthetic CRH-R1 antagonist and/or synthetic CRH-R2 agonist.

### BACKGROUND OF THE INVENTION

### 1. The Inflammatory Response

The term inflammation implies local response to noxious endogenous or exogenous stimuli causing tissue injury, characterized by capillary dilation and leukocyte infiltration and the typical signs and symptoms of inflammation which include swelling, redness, increased local and/or general temperature and pain. Endogenous and exogenous noxious agents cause inflammation including infectious diseases. Inflammation is a host defence mechanism, which might eventually harm the defending organism. High levels of these cytokines are seen in severe infectious and various inflammatory disorders. Acute or chronic inflammatory diseases of unknown etiology may be caused by a difficult to isolate infectious agent. One wellknown example is the realization that the majority of stomach ulcers are due to infection by the bacterium Helicobacter Pylori. On the other hand, diseases that are usually not associated to inflammation are actually caused by low-grade chronic inflammation. Indeed, arteriosclerosis is a characteristic example. Autoimmune diseases can also cause inflammatory reactions characteristic being the example of the immune complex deposition disease. The pro-inflammatory cytokines IL-1, TNF-alpha, and IL-6, products of stimulated macrophages play a key role in initiating the inflammatory processes. It should be noted that infectious agents might also cause acute or chronic inflammatory diseases of unknown etiology.

### 2. The CRH System

The corticotropin-releasing hormone (CRH) family of neuropeptides is composed of several members, the most prominent of which in mammals are CRH, a 41-amino acid hypothalamic peptide, and urocortin (UCN), a 40-aminoacid peptide, sharing a 45% sequence homology to CRH. The biological effects of CRH are mediated by at least two different types of receptors, the CRH-R1 and CRH-R2 that belong to the G-protein coupled receptor super-family. CRH exhibits 10 times higher affinity towards CRH-R1 compared to CRH-R2. In the immune system, CRH-R1 receptors have been identified in the spleen and thymus. The recent synthesis of non-peptide receptor antagonists for the CRH-R1 receptor provides a useful tool for a more accurate evaluation of the functional significance of CRH at the tissue level. In contrast to CRH, which has low affinity towards the CRH-R2 subtypes, UCN binds to all known effectors of CRH function, including the CRH-R1, CRH-R2α, CRH-R2β receptors and the CRH binding protein (CRH-BP).

### 3. Interaction Between CRH and Immune Systems

CRH affects the immune system directly at the site of an inflammatory reaction, and in an indirect manner via stimulation of cortisol production from the adrenals. CRH is released at the site of the inflammatory response by nerve terminals and epithelial cells directly affecting resident immune cells in the vicinity of inflammation. It should be noted that while the indirect effect of CRH is anti-inflammatory, its direct-paracrine effect is definitely pro-inflammatory. Thus, blockade of its local effect by specific anti-CRH serum, attenuates the inflammatory response in several models of inflammation in vivo. An immune target of CRH is the mast cell. However, in addition to mast cells, many other immune cells exhibit specific CRH binding sites, including mouse splenocytes, human peripheral blood monocytes, lymphocytes, monocytes-macrophages and Th cells. CRH receptors are also present in inflamed synovium and inflamed subcutaneous tissues. The CRH-R1 receptor expression in macrophages is up-regulated by exposure to Lipopolysaccharide (LPS). Indeed, the number of CRH receptors at the site of inflammation increases in parallel to CRH concentration. The role of CRH has been associated mainly to mast cells, since its administration results in mast cell degranulation, an effect inhibited by the CRH-R1 receptor antagonist antalarmin.

Macrophages are among the initiator cells during an inflammatory response and are the main source of a series of pro-inflammatory cytokines. Activation of macrophages occurs through antigenic signals such as bacterial LPS, which binds on Toll-like Receptor 4 (TLR-4) and activates cytokine transcription and secretion by these cells. During both local and systemic inflammation, macrophages are the predominant source of proinflammatory cytokines.

### 2. The CRH System in Gastrointestinal Tract (GI) Inflammation

The CRH family of peptides is expressed along the whole length of the GI tract. Indeed, CRH is produced by enterochromaffin cells in human colon while UCN is detectable in both rat stomach and colon. Recently published reports suggest that the CRH family of peptides and their receptors participate in the regulation of GI motility as well as in the GI response to inflammatory processes. Indeed, it is now well established that CRH is present in the colonic mucosa of patients with ulcerative colitis playing a local pro-inflammatory role. In addition, UCN has been identified in macrophages in the lamina propria of human colonic mucosa, participating in the regulation of the local inflammatory response. In general, it appears that the effect of the CRH family of peptides in the GI tract is receptor type specific and that the CRH-R1 and CRH-R2 receptors have more or less opposing effects. Indeed, activation of the CRH-R1 receptor results in amplification of colonic propulsive activity whereas activation of the CRH-R2 receptor results in inhibition of gastric emptying rate in mice and rats.

### DESCRIPTION OF THE INVENTION

This invention relates to the use of therapeutic modalities in the treatment of acute or chronic inflammatory diseases. The therapeutic regimens according to the invention relate to the use of synthetic CRH-R1 receptor antagonists and/or a synthetic CRH-R2 receptor agonists aiming in modifying the response of monocyte/macrophage monocyte/macrophage cell activation, proliferation, differentiation, apoptosis and cytokine production and, thus, control of the magnitude of the inflammatory response. Our data have demonstrated that CRH augments the inflammatory response, acting through the CRH-R1 receptors while UCN attenuates it acting thought the CRH-R2 receptors. These effects of CRH and UCN are the result of a direct action of these peptides on monocyte/macrophage cells which express both the CRH-R1 and the CRH-R2 receptors on the surface.

Three types of experimental models have been used to demonstrate the regulatory role of the CRH system on monocyte/macrophages: (a) in vitro cultures of macrophages, (b) in vivo animal models, and (c) a paradigm of a human inflammatory disease.

(a) In our in vitro experiments, we have used two types of macrophages, the RAW 264.7 monocyte/macrophage cell line (which derives from a mouse myeloma and produces all pro-inflammatory cytokines in response to LPS) and the thioglycollate-elicited peritoneal macrophages from Balb/c mice. CRH enhanced LPS-induced TNF-α, IL-1β and IL-6 production. On the other hand, UCN ameliorated the inflammatory response via induction of macrophage apoptosis. This effect of UCN was more pronounced in LPS-induced RAW-264.7 macrophages and primary bone marrow macrophages. Treatment of RAW264.7 cells with UCN resulted in a rapid activation of the stress-induced kinases JNK and p38MAPK, up-regulation of Bax and enhancement of Fas Ligand expression and apoptosis.

(b) In our vivo experiments, we have used the LPS-induced endotoxin shock model in Balb/c mice, an established model for systemic inflammation in which macrophages are the major source of the proinflammatory cytokines responsible for the development of the shock. We have found that administration of a synthetic CRH-R1 antagonists prior to LPS prolonged survival in a statistically significant manner. The effect was more evident at the early stages of endotoxin shock. CRH-R1 blockade suppressed LPS-induced elevation of the macrophage-derived cytokines TNF-α, IL-1β, and IL-6, confirming the role of CRH signals in cytokine expression.

(c) For our human data, we have used patients suffering from gastritis. The model of gastritis chosen in this prospective study was that caused by Helicobacter pylori (H. pylori) because of its localized and well-circumscribed nature and its complete reversibility following appropriate eradication treatment. Our fresh tissue samples were obtained from gastroscopic biopsies. The design of our study was based on our pilot data showing that the CRH transcript and peptide may not be detectable in normal human gastric mucosa, while UCN may be present and localized to gastric epithelial cells. Our data confirmed our hypothesis indicating that in human stomach UCN is a powerful suppressor of inflammation.

Thus, the inventors demonstrate both in vitro and in vivo that CRH-R1 agonists augmented the inflammatory response, CRH-R1 antagonists ameliorated it, and CRH-R2 agonists also ameliorated it. Our invention relates to the use of such compounds for the treatment of local and systemic inflammations in humans.

In one aspect the invention relates to pharmaceutical compositions comprising one or more synthetic CRH-R1 antagonists and/or CHR-R2 agonists.

The term “synthetic” in relation with the compounds according to the invention is intended to mean that the compounds in question are not naturally occurring compounds, but are manufactured using some technical processes. Synthetic compounds thus comprise e.g. proteins and peptides, provided using recombinant technologies or by chemical synthesis; and small organic compounds.

Thus a “synthetic CRH-R1 antagonist” is a synthetic compound that inhibits CRH-R1 function and when added to a CRH-R1 assay blocks the effects of CRH peptides and the effects of synthetic CRH-R1 agonists, resulting in a smaller signal when the CRH-R1 receptor is stimulated with a agonist ligand therefore, such as CRH, compared with same assay but without said compound. A “synthetic CRH-R2 agonist” is a synthetic compound that activates CRH-R2 and in a CRH-R2 assay gives rise to a signal as a result of the CRH-R2 receptor activation, such as CRH, compared with same assay but without said compound.

CRH-R1 and CRH-R2 assays are known within the art. In principle any suitable CRH-R1 and CRH-R2 assays known within the art may be used for determining if a candidate synthetic compound is an antagonist or agonist respectively. Preferred examples of CRH-R1 and CRH-R2 assays have been developed. Assays for biological activity via the CRH-R1 receptor: (a) CRH activates p38 mitogen-activated protein kinase, stimulates Fas ligand production and induces apoptosis in PC12 rat pheochromocytoma cells. The CRH-R1 antagonist antalarmin blocks all these CRH-mediated effects (Dermitzaki et al, 2002). (b) CRH enhances the inflammatory response to lipopolysaccharide (LPS) of macrophages in vitro. The enhancing effect of CRH is blocked completely by the CRH-R1 antagonist antalarmin (Agelaki et al, 2002). Assay for biological activity via the CRH-R2 receptor: Urocortin and Urocortin II induce apoptosis on macrophages. This effect is mediated by the CRH-R2 receptor since the specific antagonist sauvagine-30 completely abolishes this effect (Tsatsanis et al, submitted).

Antalarmin is an example of a synthetic CHR-R1 antagonist.

In another aspect the invention relates to the use of one or more synthetic CRH-R1 antagonists and/or CHR-R2 agonists for the manufacture of a pharmaceutical composition for the treatment of inflammatory diseases or conditions.

In addition to the active compound(s) the pharmaceutical compositions may comprise usual exhibients such as diluents, fillers, binders, disintegrants, lubricants, conserving agents, flavourings and colourings. The pharmaceutical compositions may be formulates for any suitable route of administration including oral, parenteral or intravenous administration. A preferred administration form is injection.

The amount of active compound(s) in the pharmaceutical compositions depends on the actual active compound, the age, weight, and condition of the receiver. It is within the skills of the ordinary practitioner to determine the suitable amounts of a given active compound based on routine experimentations.

The amounts to be administered and the frequency and route of administration will depend on the given compound and the actual condition to be treated and will be at the discretion of the attending physician.

Inflammatory diseases or disorders to be treated with the pharmaceutical compositions according to the invention includes but are not limited to: chronic inflammatory bowel disease, idiopathic inflammatory disorder, inflammatory disorders of connective tissues, inflammatory demyelinating polyneuropathies, inflammatory myopathies, inflammatory diseases of joints including bursitis, the fibromyalgia syndrome and inflammatory diseases of upper gastrointestinal tract.

The term “treatment” should be understood broadly and comprises in addition to treatment also prevention of a disease, alleviation of the disorder or disease and prevention of recurring of the inflammatory disease or disorder.

If more than one active compound are intended to be used for the treatment of a particular disease, according to the invention, e.g. by the use of one synthetic CRH-R1 antagonist and one synthetic CRH-R2 agonist for a particular treatment, the more that one active compound may be formulated in one pharmaceutical composition comprising all the active compounds, or the more that one active compound may be formulated in two or more different pharmaceutical compositions each comprising one or more active compound.

In case that a combination of active compounds are formulated in two or more separate pharmaceutical compositions the pharmaceutical compositions may be administrated simultaneously or they may be formulated at different point of times or frequency.

When such a combination of active compounds are formulated in two or more separate pharmaceutical compositions, these pharmaceutical compositions are suitable provided in a kit comprising one or more CRH-R1 antagonists and/or CHR-R2 agonists comprised in one of more individual pharmaceutical compositions.

Thus, in another aspect the invention relates to a kit for the treatment of inflammatory diseases or conditions comprising one or more CRH-R1 antagonists and/or CHR-R2 agonists comprised in one of more individual pharmaceutical compositions.

The kit may also contain a instruction for the frequency, amount and duration of the administration for the pharmaceutical compositions in the kit.

### DESCRIPTION OF THE DRAWINGS

FIG. 1. RAW264.7 cells were treated with 10⁻⁹M UCN and 10 μg/ml LPS and apoptosis was measured by nucleosome formation.

FIG. 2. UCN enhances LPS-induced p38MAPK and JNK activation in RAW264.7 macrophages

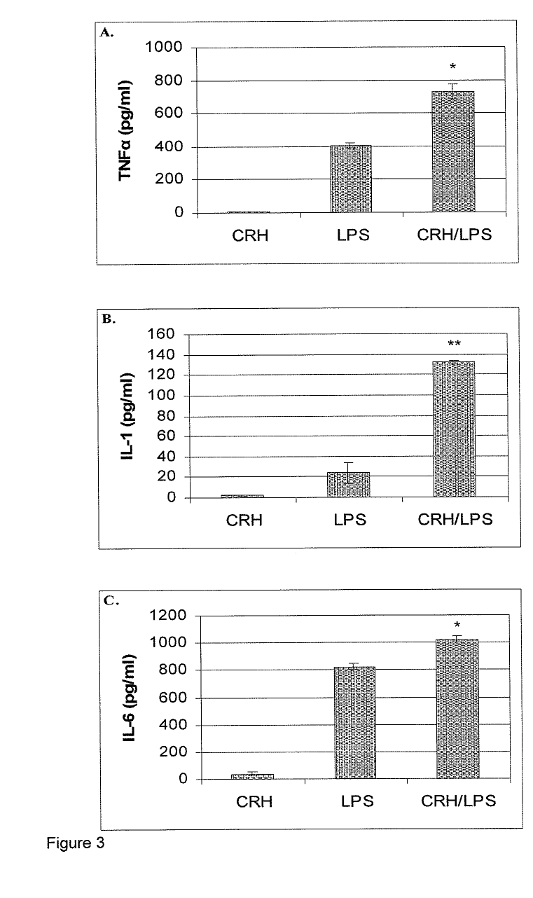

FIG. 3. CRH augments LPS-induced pro-inflammatory cytokine secretion from RAW264.7 macrophages. a) TNF-α levels in the culture medium of cells treated with CRH, LPS and CRH plus LPS. TNF-α levels are significantly higher when cells are treated with CRH and LPS than LPS alone. b) CRH potentiates LPS-induced IL-1β secretion in a significant manner. c) CRH potentiates LPS-induced IL-6 secretion from RAW264.7 cells.

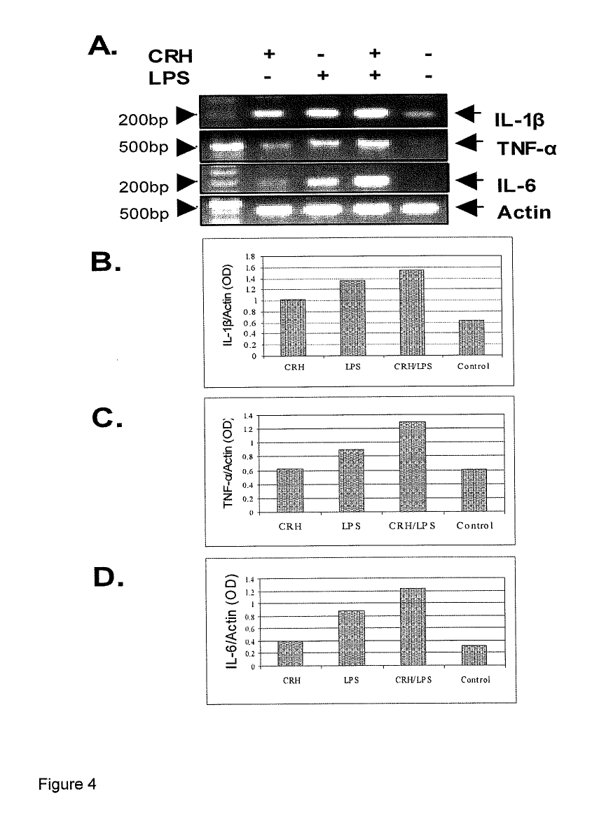

FIG. 4. a) CRH augments pro-inflammatory cytokines at the transcriptional level. IL-1β (upper panel), TNF-α (second panel) and IL-6 (third panel) mRNA levels were determined by a semi-quantitative RT-PCR approach. CRH induces expression of all three cytokines and further potentiates the LPS-induced transcriptional activation. b, c, d) Densitometric analysis of the RT-PCR products of IL-1β (B), TNF-α (C) and IL-6 (D).

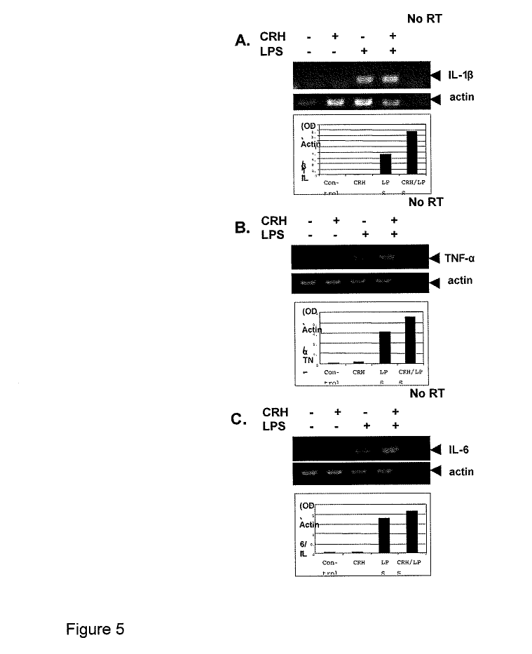

FIG. 5. CRH augments LPS-induced pro-inflammatory cytokine expression in thioglycollate-induced peritoneal macrophages from Balb/c mice. IL-1β (A upper panel), TNF-α (B upper panel) and IL-6 (C upper panel) mRNA expression was quantitated by densitometry of RT-PCR products normalized per actin.

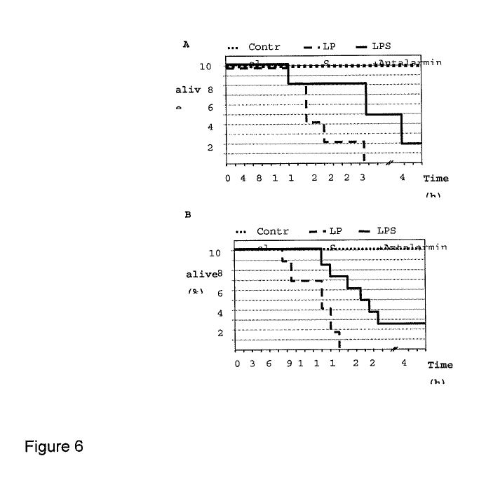

FIG. 6. The CRH-R1 receptor antagonist antalarmin prolonged survival of Salmonella enteritidis-derived LPS treated animals.

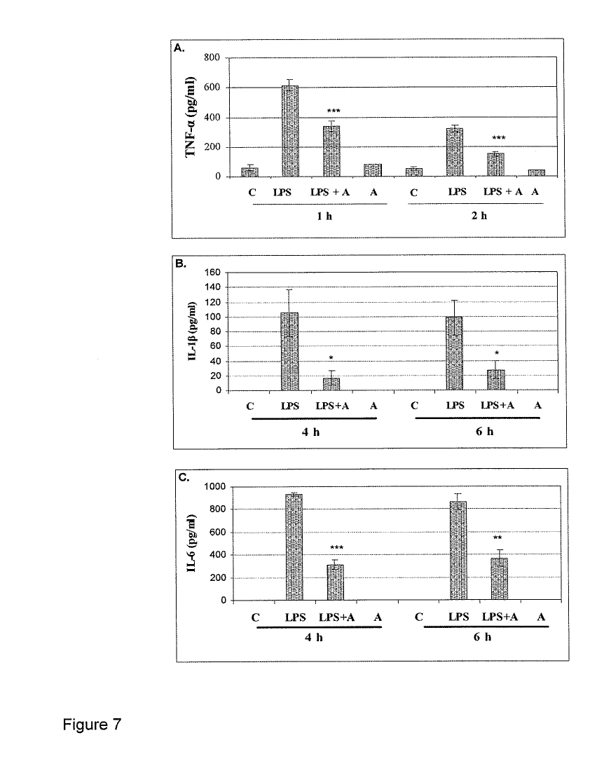

FIG. 7. Blockade of the CRH-R1 receptors by Antalarmin significantly reduced TNF-α. IL-1β, and IL-6 levels in mice subjected to LPS-induced endotoxin shock.

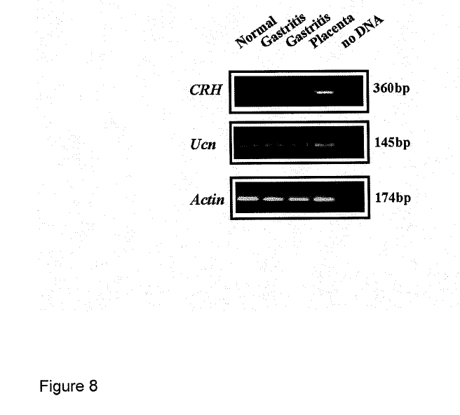

FIG. 8. RT-PCR analysis for CRH-like peptides in total RNA isolated from a biopsy of human normal gastric mucosa, two biopsies of inflamed gastric mucosa from two different subjects with gastritis, and of human placenta at term. The predicted size DNA product of 145 bp for UCN is found in all samples. Negative control samples are also shown with no reverse transcriptase enzyme (noRT), or no DNA template. The predicted size DNA product of 360 bp for CRH was detected only in the placenta RNA sample. RT-PCR for actin was performed to assure RNA quality in all samples.

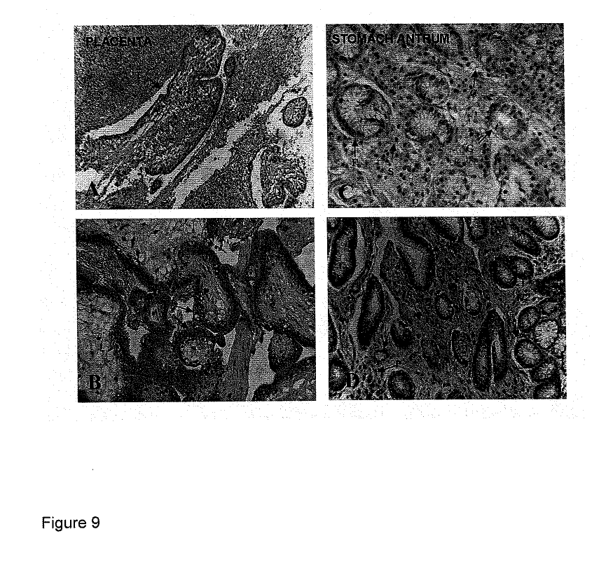

FIG. 9. Immuno-histo-chemical staining for UCN in gastric mucosa from patients with chronic gastritis associated with Helicobacter pylori infection (C, D). Human placenta was used as positive control (Panels A, B). Gastric mucosa and placental tissue sections were stained with anti-UCN antibody (Panels B, D). Immunoreactive Urocortin (Ir-UCN) was localized to the epithelial cells of the faveolars (F) and to the mucus secreting glands (G). Positive staining was also observed in the capillaries (C) and in inflammatory elements scattered of the gastric mucosal stroma (S), mostly plasma cells (P). In the placental sections, trophoblastic epithelial cells (T) stained positively for UCN in contrast to the adjacent stroma villii (V). Control immunostaining using normal rabbit IgG or UCN peptide-inactivated antibody (Panels A, C) was uniformly negative. Original magnification x250.

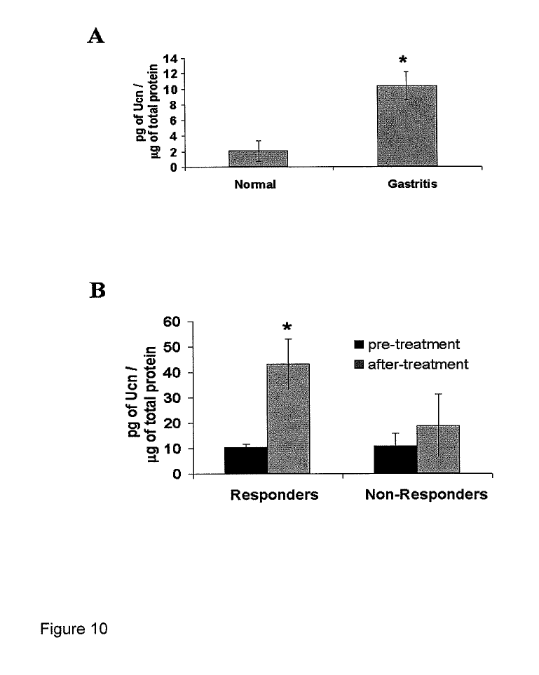

FIG. 10. Levels of UCN in human gastric mucosa biopsies. Panel A. Comparison between patients with no gastric inflammation (normal) and patients diagnosed for gastritis due to Helicobacter pylori infection. Ir-UCN was found significantly elevated (p<0.001) in the group of patients with H. pylori gastritis and gastric inflammation. Panel B. Comparison between patients with H. pylori gastritis before treatment and two months after receiving medication for H. pylori eradication. According to pathology findings, the latter was subdivided into responders (regression of acute and chronic inflammation and no signs of H. pylori infection) and non-responders (persisting inflammation and/or signs of H. pylori infection). A significant increase of ir-UCN levels was noted in the group of treated (p<0.001) but not in the non-responding patients.

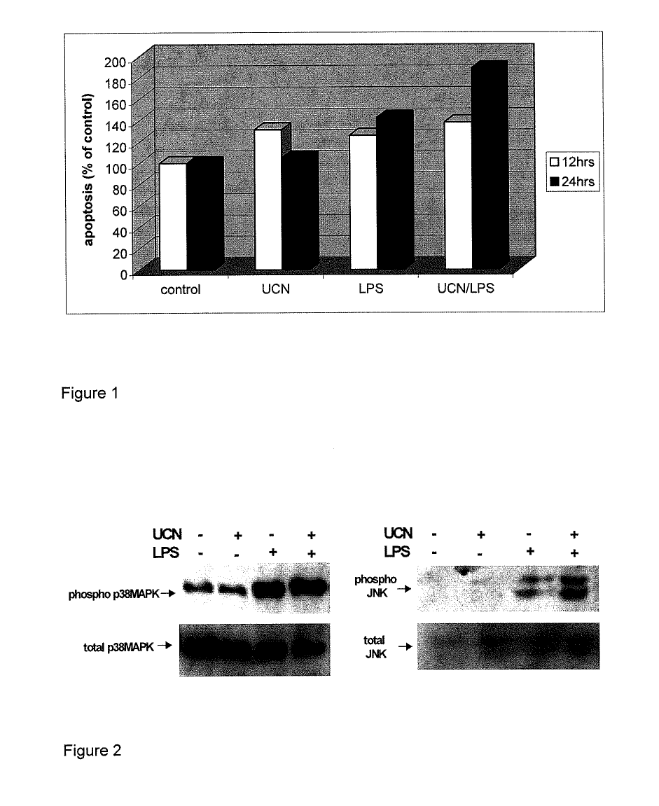

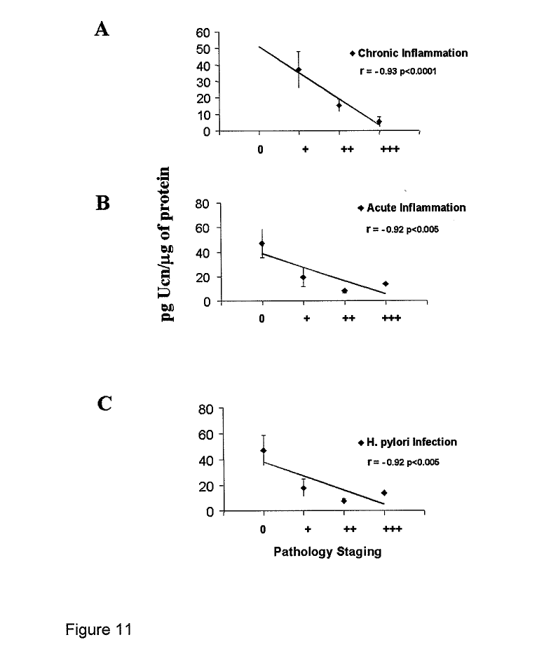

FIG. 1. Correlation analysis between UCN levels and levels of inflammatory activity in gastric biopsies from patients with gastritis (n=30). A: chronic inflammation B: acute inflammation and C: H. pylori infection. A statistically significant negative correlation was observed with all three parameters (Spearman's rank correlation).

### EXPERIMENTAL

The invention is now further described by way of experimental works, which should not be considered as limiting in any way.

Materials and Methods

Cell Cultures

RAW 264.7 cells were cultured in Dulbecco's Modified Eagle Medium supplemented with 10% fetal calf serum (FCS), 10 mM L-glutamine, 100 U/ml penicillin, 0.1 mg/ml streptomycin (all purchased by Gibco), at 5% CO₂ and 37° C. Cells were plated in 25 cm² flasks one day prior to stimulation. Cells were then stimulated with 10 μg/ml Ecoli-derived LPS (serotype 0111:B4, cat. # L2630, Sigma) and recombinant CRH (Sigma) at a concentration of 10⁻⁸ M.

Isolation and Stimulation of Thioglycollate-Elicited Macrophages

A 4% thioglycollate solution was prepared and autoclaved 2 days prior to administration. 1.5 ml of the thioglycollate solution was injected intraperitoneally in BALB/c mice and peritoneal macrophages were isolated by lavage of the peritoneal cavity with Dulbecco's Modified Medium. Cells were then cultured in DMEM supplemented with 10% FCS, 10 mM L-glutamine, 100 U/ml penicillin, 0.1 mg/ml streptomycin (Gibco). Cells were plated at a concentration of 5×10⁵/ml and maintained in culture 24 hours prior to stimulation.

Animals

Male 20- to 25-g Balb/c mice 8 to 10 weeks old were used. They were kept in our animal facility for at least one week prior to each experiment to allow adjustment and confirmation of their health. Each animal received rodent laboratory chow and water ad libitum. The CRH-R1 antagonist used was provided by the Pediatric and Reproductive Endocrinology Branch, NICHD, NIH, Bethesda, Md. Antalarmin was initially dissolved into 100% ethanol at a concentration of 200 mg/ml and then diluted in a 1:1 ratio with Cremaphor EL (Sigma), and finally brought to a working stock of 2 mg/ml antalarmin in 10% ethanol and 10% Cremaphor EL in sterile water. E coli lipopolysaccharide (serotype 0111:B4, cat. # L2630) and Salmonella enteritidis lipopolysaccharide (cat. # L6011) were purchased from Sigma. The antibodies and the reagents for the TNF-α, IL-1β and IL-6 determination were purchased from R&D (NE, USA).

LPS-Induced Endotoxin Shock

For the determination of the LD₅₀ groups composed of five mice were injected intraperitoneally (i.p) with either 200, 400, 600, 700 or 1000 μg/mouse of Salmonella-derived LPS (Sigma) dissolved in PBS at a concentration of 10 mg/ml. Survival of animals was monitored for a period of seven days. The same protocol was used for E. coli-derived LPS (O111:B4). To determine the effect of antalarmin in the survival of mice injected with LPS, 40 mice were divided in four different groups; the first group received antalarmin at a concentration of 20 mg/kg of body weight; the second received antalarmin at 20 mg/kg of body weight and LPS at a concentration of 0.7 mg per 25 g of body weight; the third group received LPS and the antalarmin diluent while the fourth group received the antalarmin diluent alone. Mice were pre-treated with antalarmin or the diluent 1.5 hours prior to LPS injection, according to protocols and in order not to alter significantly the HPA axis response. The CRH-R1 receptor antagonist Antalarmin alone had no effect in the survival of animals and injection of antalarmin alone was not repeated in the course of the experiments.

Isolation of Total RNA and RT PCR

Total cellular RNA was isolated using Trizol reagent (Gibco). Following reverse transcription (Thermoscript RT, Invitrogen), 1 μl of the cDNA product was amplified by PCR at 33 cycles. It should be noted that at 33 cycles all mRNA amplifications were at the exponential phase of amplification as indicated by a standard curve performed for each pair of primers (data not shown). 10 μl of the amplified products were separated on a 3% agarose gel and visualized by ethidium bromide staining. Intensity of the bands was quantified using TINAscan software.

Primers for actin were: sense, 5′-TCA GAA GAA CTC CTA TGT GG-3′; antisense, 5′-TCT CTT TGA TGT CAC GCA CG-3′, giving a 499 bp product; for Tnf-α were: 5′-CAC GCT CTT CTG TCT ACT GAA CTT CG-3′; 5′-GGC TGG GTA GAG AAT GGA TGA ACA CC-3′, giving a 590 bp product; for IL-1β were 5′-GGA TGA GGA CAT GAG CAC CT-3′ and 5′-TCC ATT GAG GTG GAG AGC TT-3′, resulting in a 196 bp product; for IL-6,5′-TGA AGT TCC TCT CTG CAA GAG ACT-3′,5′-TGA GGA AGG CCG TGG TTG T-3′, giving a 200 bp product. Total cellular RNA was isolated using Trizol reagent (Gibco). Following reverse transcription (Thermoscript RT, Invitrogen), 1 μl of the cDNA product was amplified by PCR (Platinum Taq polymerase, Invitrogen), at 33 cycles, annealing to temperature of 55° C. It should be noted that at 33 cycles all mRNA amplifications were at the exponential phase of amplification as indicated by a standard curve performed for each pair of primers (data not shown). 10 μl of the amplified products were separated on a 3% agarose gel and visualized by ethidium bromide staining using the BioRad Molecular Analyst System. The quantitation was performed using the “TI-NAscan” software. Each experiment was repeated four times.

Total tissue RNA was extracted from frozen biopsies of stomach antrum using the Trizol Reagent (Gibco BRL Co, MD). Contaminant genomic DNA was removed by the addition of DNase (Gibco BRL). Reverse transcription was performed using the SuperScript Preamplification System (Gibco BRL) and random hexamers in a total volume of 20 μl. 2 μl of the RT product was used as template, amplified by PCR using 2 mM MgCl₂, one strength PCR buffer, 0.2 mM of sense and antisense primers, 0.2 mM dNTPs and 2.5 U Taq Polymerase (Gibco) in a final reaction volume of 50 μl. PCR was performed in a Perkin-Elmer DNA Thermal Cycler with the following cycling parameters: a pre-amplification cycle (denaturation for 5 min at 98° C., annealing for 1 min at 65° C. and extension for 1 min at 72° C.), 2 cycles with annealing for 1 min at 63° C., 35 cycles of amplification (denaturation for 1 min at 95° C., annealing for 1 min at 60° C. and extension for 1 min at 72° C. and a final extension step for 7 min at 72° C. The oligonucleotides were designed as per the published human sequences for Ucn sense: 5′-CAGGCGAGCGGCCGCG-3′, and anti-sense: 5′-CTTGCCCACCGAGTCGAAT-3′ and CRH sense: 5′-CAACTTTTTCCGCGTGTTGCT-3′, and anti-sense: 5′-ATGGCATAAGAGCAGCGCTAT-3′. The size of the amplified product was expected to be 145 bp for Ucn and 360 bp for CRH. Oligonucleotides were custom-synthesized by MWG-Biotech, AG (Munchen, Germany). Negative control samples where no RT enzyme was added in a positive for Ucn RNA sample (noRT) or without DNA template (no DNA) were included in every assay in order to exclude the possibility of genomic or other DNA contamination. PCR for actin was also performed using a standard procedure to assure good quality of RNA and cDNA preparations. The amplified PCR products were fractionated by 1% agarose gel electrophoresis, detected by ethidium bromide staining under UV.

ELISA and RIA

Serum from trunk blood was collected as follows: a) for the determination of TNF-α 1 hour after LPS administration and b) at 4 hours for the determination of IL-1β or IL-6 levels. Each time point and treatment group was composed of five animals per experiment. Sera were collected and frozen until used for cytokine determination by ELISA according to manufacturer (R&D, NE, USA). Similarly, cell culture supernatants were collected 24 hours following stimulation and stored at −70° C. until analyzed. Corticosterone was measured by RIA in serum collected 1 hour following LPS administration. Five animals per treatment were used. Sera were frozen at −70° C. and analyzed as recommended by the manufacturer (ICN, USA).

Quantitative Measurement of Apoptosis

Cells were plated in 96-well plates at an initial concentration of 10,000 cells per well. Apoptosis was measured by direct determination of nucleosomal DNA fragmentation with the “cell death detection” ELISA plus kit according to the manufacturer's protocol.

FACS Analysis

Cells were treated with UCN and/or LPS at the indicated concentrations for different time points, collected in PBS and incubated with 7-amino-actinomycin (7AAD) for 10 minutes. Cells were washed and analyzed by Flow Cytometry (Coulter).

Western Blot Analysis

Following stimulation cells were harvested and lyzed in 62.5 mM Tris HCl pH=6.8, 10% glycerol, 2% SDS, and freshly added inhibitors phenylmethylsulfonyl fluoride (10 μg/ml), 0.5 mM DTT and 50 mM Na₂F.

Human Tissue Specimens

Patients with a history of epigastric pain and/or dyspeptic symptoms of more than one-month duration underwent gastroscopy as per existing diagnostic protocols in effect at the University Hospital in Heraklion, Crete. The following categories of patients were exclude: those with a history of duodenal or gastric ulcer, esophagitis either as a result of gastroesophageal reflux or infection, motility disorders of the esophagus and upper GI tract, gallbladder or bile duct lithiasis, pancreatitis, cirrhosis, inflammatory bowel disease (Crohn's disease and ulcerative colitis), diabetes mellitus or cancer. Excluded were also patients that were taking any medicine except antacids during the previous month. Following careful exclusion of all the above-mentioned cases, patients that underwent gastroscopy were divided into two groups: controls (n=8) and patients with diagnosed H. pylori gastritis (n=15). Between the two groups there were no differences regarding age, gender, dietary habits, or smoking. As controls were defined patients having normal endoscopic findings and absence of inflammatory elements in antral biopsies. As patients with chronic gastritis due to Helicobacter pylori were defined individuals with compatible history and diagnostic findings including flat or raised erosions of gastric mucosa and exudative elements. The lesions were usually more prominent in gastric antrum. The presence of H. pylori was detected histologically and by placement of antral biopsies in a urea-containing medium allowing simple detection from the pH change that occurs as ammonia is produced by the organism (CLO-test, Delta West, Bentley, Australia). A second gastroscopy was performed two months after eradication treatment, consisting of a double antibiotic 10-day scheme (amoxicillin 1 g P.O. b.i.d, clarithromycin 500 mg P.O. b.i.d.) and omeprazole (20 mg P.O b.i.d. for 10 days and then 20 mg q.d. for a month). To attain a more representative measurement of immunoreactive urocortin (ir-Ucn) levels in stomach antrum, samples were collected from antrum (lesser and greater curves, front and back wall) by endoscopic biopsy forceps. Samples of each patient were pooled and frozen immediately at −70° C. The histological grading of gastritis was based on the Sydney classification and was performed by the same person, not aware of the different groups of patients. Chronic inflammation was graded as mild, moderate and severe and was noted by (+), (++) and (+++) respectively. The absence of any inflammation was indicated as zero. The presence of Helicobacter pylori was graded with (+), (++) and (+++) depending on the density of its presence on the superficial epithelium of antrum mucosa. As normal biopsies, were considered those with absent inflammation and negative for H. pylori. Human term placenta was obtained from women undergoing labor at the Obstetrics and Gynecology Department, Heraklion University Hospital. Informed consent and full ethics committee approval had been obtained prior to the collection of the specimens.

RIA for Urocortin

Pooled gastroscopic antral biopsy samples from each patient were homogenized in ice-cold 0.1 N HCl and centrifuged at 10,000 g for 20 min at 4° C. The supernatants were acidified by 10 volumes of 0.1 N HCl, centrifuged at 10,000 g for 10 min, and the new supernatants were extracted by activated Sep-Pak C18 cartridges column (Sep-Pak, Waters Associates, Milford, Mass.), washed with 20 ml 0.1 N HCl, eluted with 3 ml 80% acetonitrile 20% 0.01N HCl and dried under vacuum (Speed-Vac). Ir-Ucn was assayed by a RIA Ucn kit (Peninsula Laboratories, Inc., CA, USA) following manufacturer's instructions. The rabbit antiserum used (RIK 8034) exhibits 100% cross-reactivity to human Ucn and no cross-reactivity to human CRH, urocortin II and III, sauvagine, urotensin I and II. In our hands, the sensitivity of the assay was 10 pg and the IC50 109 pg/tube. Results were expressed as pg of ir-CRH per μg of total cellular protein determined on whole cellular homogenates by the Bradford method.

Immunohistochemistry

Immunostaining was performed on formalin-fixed, paraffin-embedded tissue sections. Paraffin sections from biopsies of stomach antrum were cut and stained by the standard alkaline phosphatase—antialkaline phosphatase (APAAP) method (DAKO, A/S, Glostrup, Denmark). Briefly, paraffin sections were heated in microwave in a solution of sodium citrate prior to incubation with the antibodies. Xylene dewaxed and alcohol rehydrated sections were placed in coplin jars filled with a 0.1M trisodium citrate solution and heated three times in a conventional microwave oven for 5 min at 700 W. Slides were then allowed to cool for 15 min and washed in Tris buffered saline (TBS). After blocking non-specific binding sites by incubation with normal rabbit serum (1:20, 30 min, RT) in a humid chamber, first antibody incubation (1 h, RT), using the rabbit anti-UCN polyclonal serum described above (IHC 8034, Peninsula Laboratories, Inc., dilution 1:1000). After washing with TBS, sections were incubated with anti-rabbit IgG and the APAAP complex (DAKO). Fast red TR (DAKO, A/S, Glostrup, Denmark) was used as a chromogen with a light haematoxylin counterstain and sections were mounted with warm glycergel (DAKO). Negative control sections were included in each experiment by incubation with non-immune IgG instead of the primary antibody or using antiserum inactivated by 1 μM UCN peptide (Sigma, St. Louis, Mo., USA) over night at RT. Photographs were taken in a standard light microscope using Kodak Elite Chrome film 100 ASA.

Results

A. In Vitro Studies

Effect of UCN on Macrophages

UCN promoted apoptosis in serum-deprived, LPS-induced RAW-264.7 macrophages as determined by nucleosome formation and Flow Cytometric Analysis. When cells were cultured in the presence of serum, UCN promoted cell proliferation. The same effects were observed on primary bone marrow macrophages where UCN enhanced LPS-induced apoptosis.

Molecular Mechanisms Through which UCN Promotes Macrophage Apoptosis

Treatment of RAW264.7 cells with UCN resulted in a rapid activation of the cellular stress-induced kinases JNK and p38MAPK, upregulation of Bax and enhancement of Fas Ligand expression.

CRH Enhances LPS-Induced Cytokine Production from RAW264.7 Cells

To determine the effect of CRH on macrophages, RAW264.7 cells were cultured in media containing serum and stimulated with Ecoli-derived LPS in the presence or absence of CRH at a concentration of 1×10⁻⁸ M. The concentration used is within the physiological range for CRH in peripheral tissues since in the placenta it is found at a concentration of 10⁻⁶ M and in the adrenals it can vary between 10⁻⁶ to 10⁻⁹ M. Treatment of cells for 24 hours in the presence of LPS stimulated the secretion of TNF-α, IL-1β and IL-6. In the presence of CRH the levels of all three cytokines were significantly higher, indicating that CRH augments the LPS signal. However, there was only a minimal effect on cytokine secretion when cells were treated with CRH alone. Specifically, CRH significantly augmented LPS-induced TNF-α secretion (p=0.04), IL-1β secretion (p=0.01) and IL-6 secretion (p=0.04).

To determine whether CRH has an effect on cytokine transcription, RNA was isolated from cells treated with LPS in the presence or absence of CRH and the levels of TNF-α, IL-1β and IL-6 mRNA was estimated using a semi-quantitative RT-PCR approach. The PCR reactions were carried out at 33 cycles where the amplification was at the exponential phase, as determined by the curve of the amplification of each product. CRH has a minor enhancing effect at the basal mRNA levels of all three cytokines and a stronger augmenting effect on the LPS-induced levels. Densitometric analysis of the RT-PCR products following normalization versus actin revealed that CRH alone induced minimal transcription of IL-1β, TNF-α, or IL-6, but it strongly augmented the LPS-induced cytokine transcription. The increase that occurred at the transcriptional level was lower to the increase indicated at the protein level suggesting that there may be an additional effect of CRH at the posttranscriptional level. Alternatively this may be the result of the lower sensitivity of the semi-quantitative approach of RT-PCR. The same experiment was repeated four times with similar results.

CRH Enhances LPS-Induced Cytokine Production in Thioglycollate-Elicited Peritoneal Macrophages.

To determine whether CRH exerts the same effect in primary macrophages, we treated thioglycollate-induced peritoneal macrophages with CRH and CRH plus LPS. Thioglycollate-induced macrophages are primed inflammatory macrophages and using this approach one could study inflammatory macrophages without having to accelerate them with LPS. CRH was unable to induce TNF-α, IL-1β or IL-6 transcription but significantly augmented the LPS-induced pro-inflammatory cytokine expression. The densitometric data were analyzed and showed similar differences with the ones observed in RAW264.7 cells. Thus, CRH has a potent effect in both activated RAW264.7 cells and activated primary macrophages and it cannot elicit cytokine expression in the absence of a potent co-stimulus such as LPS.

B. In Vivo Studies

The CRH-R1 Antagonist Antalarmin Prolonged Survival of Mice Subjected to LPS-Induced Septic Shock

LPS was administered i.p. at the concentrations of 0.2, 0.4, 0.6, 0.7 and 1 mg per 25 g of body weight. One hundred percent of animals treated with LPS at 0.2 mg/25 g survived compared to 80% of the animals treated at 0.4 mg/25 g, 40% at 0.6 mg/kg and none of the animals survived at 0.7 or 1 mg/25 g. The LD₅₀ was estimated at 0.5 mg per 25 g body weight and the LD₁₀₀ at 0.7 mg per 25 g of body weight and over. For the purpose of our experiment we wanted to use a higher dose than the LD₅₀ to determine the possible protective effect of CRH-R1 blockade. Thus, mice were injected with 0.7 mg/25 g of LPS, an LD₁₀₀ dose but not too high to mask a possible protective effect of CRH-R1 blockade. To determine the role of CRH-R1 signals in the cascade of events that take place during septic shock, mice were subjected to a lethal dose of LPS with or without IP administration of the CRH-R1 antagonist antalarmin 1.5 h prior to the administration of LPS, to ensure absorbance according to previous reports. Two different types of LPS were used to confirm that the results where not specific to a particular type of LPS. Intra-peritoneal injection of LPS at a dose of 0.7 mg per 25 g of bodyweight induced lethality within 12 to 31 hours after injection. Specifically, in mice treated with Salmonella enteritidis-derived LPS alone lethality was observed between 14 to 31 h. At 18 hours 60% of the animals had died compared to only 20% of the mice pretreated with the CRH-R1 antagonist antalarmin. Overall, survival was significantly prolonged in the mice pre treated with the CRH-R1 antagonist antalarmin (p=0.022). Similarly, 72% of the injected with E coli-derived LPS mice and pre-treated with the CRH-R1 antagonist antalarmin were still alive at 18 h while all the animals treated with Ecoli-LPS alone had died. Mice that were treated with LPS plus the CRH-R1 antagonist antalarmin and survived the endotoxin shock were observed over a period of 7 days and were still alive indicating that treatment with the CRH-R1 antagonist antalarmin not only prolonged but also improved the survival. All animals treated with the CRH-R1 antagonist antalarmin alone survived. The overall survival was significantly improved in the presence of the CRH-R1 antagonist antalarmin (p=0.002). The experiment was repeated 3 times for each LPS subtype using 10 animals per group.

The CRH-R1 Antagonist Antalarmin Suppressed Endotoxin-Induced Proinflammatory Cytokines

LPS administration resulted in an acute elevation of plasma TNF-α peaking at 1 h. TNF-α was significantly reduced in mice pre-treated with antalarmin compared to LPS alone (n=5 animals per group, p=0.001). Similarly, plasma IL-1β and IL-6 reaches a peak 3-4 hours following LPS treatment and remains elevated throughout the septic shock. Both IL-1β and IL-6 increased at 4 hours following LPS administration but were significantly lower in mice that were pre-treated with antalarmin (n=5 animals per group, p=0.013, for IL-1β; n=5 animals per group, p<0.001, for IL-6). To determine whether the difference in cytokine levels in the presence of antalarmin is a result of a change in kinetics, we measured TNF-α at 2 hours following LPS injection and found that the levels of TNF-α in the mice that were pre-treated with antalarmin remained significantly lower than in the animals treated with LPS alone (p<0.001). Similar differences were observed when measuring IL-1β and IL-6 six hours following LPS injection. Thus, LPS treated animals had significantly higher levels of IL-1β (p<0.01) and IL-6 (p<0.001) than LPS plus antalarmin treated mice at 6 hours. We could, therefore, conclude that antalarmin prolonged survival during LPS-induced septic shock by lowering pro-inflammatory cytokine levels rather than altering their kinetics.

The UCN Transcript is Present in Normal and Inflamed Human Gastric Mucosa

Expression of CRH-like peptides was studied in antral biopsies from human gastric mucosa, using RT-PCR in total RNA preparations. Using primers designed to target the human UCN gene, a unique RT-PCR product was amplified in RNA preparations from biopsies of both normal and inflamed gastric mucosa. The size of the DNA band was the same as the one amplified from a human placenta RNA sample used as a positive control. No PCR product was detected in the negative control samples performed in parallel, using no reverse transcriptase enzyme or no cDNA template, excluding the possibility of genomic or other DNA contamination of the samples. In contrast, when RT-PCR was performed using primers for the human CRH gene, no PCR products were amplified in the RNA samples from normal and inflamed gastric mucosa, as in the placenta sample that resulted in a DNA band of the predicted size. The quality of the RNA preparations from these samples was assured by RT-PCR amplification for the actin gene. These results revealed the presence of the UCN but not the CRH gene transcripts in the gastric mucosa of the human stomach.

The UCN Peptide in Normal and Inflamed Human Gastric Mucosa.

UCN was present in the epithelial cells of the faveolars and the mucus secreting glands (antral glands) in patients with H. pylori infection. Positive staining was also localized to the capillaries and to inflammatory elements scattered in the gastric mucosal stroma, mostly plasma cell aggregates. Human term placenta was stained as positive control. In the placental sections specific positive staining was observed in the trophoblastic epithelial cells in contrast to the adjacent stroma negative villii, confirming specificity of the method. Replacement of the primary antibody by non-immune IgG or inactivation of the antibody by excess UCN peptide before the procedure resulted in uniformly negative immunostaining in both tissue types.

Relationship of UCN Levels to Inflammation in Gastric Mucosa.

The levels of ir-UCN in the biopsies of human gastric mucosa were correlated to the degree of local inflammatory activity. Patients were grouped as follows: a) subjects with no active gastritis, i.e. no evidence of acute or chronic inflammation or H. pylori infection (n=8), b) patients with diagnosed gastritis with chronic and acute inflammation of the gastric mucosa and H. pylori infection (n=15), c) responders following a two months therapy for H. pylori eradication with pathologically confirmed regression of inflammation and no elements of H. pylori infection (n=10) and d) non-responders with remaining elements of inflammation (chronic or acute) and H. pylori infection (n=5). UCN was found significantly elevated (p<0.001) in the group of H. pylori gastritis patients (group b, 10.4±1.8 pg/μg of total protein) compared to non-gastritis subjects (group a, 2.0±1.3 pg/μg of total protein). The UCN levels were further increased in the group of responders to the treatment of H. pylori eradication (group c, 43.1±9.8 pg/μg of total protein, P<0.001) compared to all other groups (a, b and d). It must be noted that no such elevation was observed in the group of non-responders to the treatment (c, 18.7±12.3 pg/μg of total protein). Correlation of the RIA data from the gastritis biopsies (n=30) with each of the pathology parameters examined (acute and chronic inflammation and degree of H. pylori infection) revealed a significant negative correlation between the levels of UCN and the pathological staging of gastritis by means of both acute and chronic inflammation and H. pylori infection, confirming an increase in the ir-UCN levels during regression of the inflammatory activity and H. pylori infection.

In conclusion, this invention provides pharmacological means of controlling inflammation by using novel and hitherto unsuspected means involving pharmacological manipulation of tissue CRH system which controls monocyte/macrophage activation, proliferation, differentiation, apoptosis and cytokine production. We have found that CRH augments the inflammatory response while UCN attenuates it. These effects of CRH and UCN are the result of a direct action on monocyte/macrophage cells. In our in vivo experiments, for which we have used the LPS-induced endotoxin shock model in Balb/c mice (an established model for systemic inflammation) administration of synthetic CRH-R1 antagonists prior to LPS prolonged survival in a statistically significant manner. The effect was more evident at the early stages of endotoxin shock. CRH-R1 blockade also suppressed LPS-induced elevation of the macrophage-derived cytokines TNF-α, IL-1β, and IL-6, confirming the role of CRH-mediated signals in cytokine expression. In our in vitro experiments, for which we have used two types of macrophages, the RAW 264.7 monocyte/macrophage cell line (which derives from a mouse myeloma and produces all pro-inflammatory cytokines in response to LPS) and the thioglycollate-elicited peritoneal macrophages from Balb/c mice, we have found that CRH enhanced LPS-induced TNF-α, IL-1β and IL-6 production. Thus, CRH signals play an early and crucial role in augmenting LPS-induced pro-inflammatory cytokine production by macrophages. We have also found that UCN ameliorated the inflammatory response via induction of macrophage apoptosis. This effect of UCN was more pronounced in vitro in LPS-induced RAW-264.7 macrophages and in primary bone marrow macrophage cultures. Treatment of RAW264.7 cells with UCN resulted in a rapid activation of stress-induced kinases JNK and p38MAPK, up-regulation of Bax and enhancement of Fas Ligand expression and apoptosis. Furthermore, our findings obtained in animals both in vitro and in vivo were also confirmed in humans. Indeed, in gastric mucosal biopsies from patients with Helicobacter Pylori gastritis, eradication treatment of HP resulted in a dramatic increase of UCN, compared to non-responding patients where UCN levels remained low further confirming the cytoprotective role of UCN in gastric mucosa against noxious stimulants including Helicobacter pylori infection. Thus, our combined data suggest that the CRH UCN system plays an important role in the regulation of the inflammatory response via its effects on monocyte/macrophage proliferation, differentiation, apoptosis and inflammatory cytokine production.

### REFERENCES

Below are listed the references cited in the description and examples. These references are considered included in the description by reference.
Agelaki S, Tsatsanis C, Gravanis A, Margioris A N. Corticotropin-releasing hormone augments proinflammatory cytokine production from macrophages in vitro and in lipopolysaccharide-induced endotoxin shock in mice. Infect Immun. 2002; 70:6068-6074.
Agnello D, Bertini R, Sacco S, Meazza C, Villa P, Ghezzi P. (1998) Corticosteroid-independent inhibition of tumor necrosis factor production by the neuropeptide urocortin. Am J. Physiol. 275:757-762.
Audhya, T., R. Jain, C. S. Hollander. (1991). Receptor-mediated immunomodulation by corticotropin-releasing factor. Cell Immunol 134:77-84
Baigent, S. M., and P. J. Lowry. (2000). mRNA expression profiles for corticotrophin-releasing factor (CRF), urocortin, CRF receptors and CRF-binding protein in peripheral rat tissues. J Mol Endocrinol 25:43-52.
Bamberger C M, Bamberger A M. (2000) The peripheral CRH/urocortin system. Ann N Y Acad Sci 917:290-296
Bamberger C M, Wald M, Bamberger A M, Ergun S, Beil F U, Schulte H M. (1988) Human lymphocytes produce urocortin, but not corticotropin-releasing hormone. J Clin Endocrinol Metab. 83:708-711.
Brar B K, Stephanou A, Okosi A, Lawrence K M, Knight R A, Marber M S, Latchman D S. (1999) CRH-like peptides protect cardiac myocytes from lethal ischaemic injury. Mol Cell Endocrinol 158:55-63
Crofford, L. J., H. Sano, K. Karalis, E. L. Webster, E. A. Goldmuntz, G. P. Chrousos, and R. L. Wilder. (1992). Local secretion of corticotropin-releasing hormone in the joints of Lewis rats with inflammatory arthritis. J Clin Invest 90:2555-64.
Crofford, L. J., H. Sano, K. Karalis, T. C. Friedman, H. R. Epps, E. F. Remmers, P. Mathern, G. P. Chrousos, and R. L. Wilder. (1993). Corticotropin-releasing hormone in synovial fluids and tissues of patients with rheumatoid arthritis and osteoarthritis. J Immunol 151:1587-96.
Dautzenberg F M, Hauger R L. (2002) The CRF peptide family and their receptors: yet more partners discovered. Trends Pharmacol Sci 23:71-77
Dermitzaki, E., C. Tsatsanis, A. Gravanis, and A. N. Margioris. (2002). Corticotropin-releasing Hormone Induces Fas Ligand Production and Apoptosis in PC12 Cells via Activation of p38 Mitogen-activated Protein Kinase. J Biol Chem 277:12280-7.
Elenkov, I. J., and G. P. Chrousos. (1999). Stress Hormones, Th1/Th2 patterns, Pro/Anti-inflammatory Cytokines and Susceptibility to Disease. Trends Endocrinol Metab 10:359-368.
Elenkov, I. J., E. L. Webster, D. J. Torpy, and G. P. Chrousos. (1999). Stress, corticotropin-releasing hormone, glucocorticoids, and the immune/inflammatory response: acute and chronic effects. Ann N Y Acad Sci 876:1-11; discussion 11-3.
Karalis, K., H. Sano, J. Redwine, S. Listwak, R. L. Wilder, and G. P. Chrousos. (1991). Autocrine or paracrine inflammatory actions of corticotropin-releasing hormone in vivo. Science 254:421-3.
Kawahito, Y., H. Sano, S. Mukai, K. Asai, S. Kimura, Y. Yamamura, H. Kato, G. P. Chrousos, R. L. Wilder, and M. Kondo. (1995). Corticotropin releasing hormone in colonic mucosa in patients with ulcerative colitis. Gut 37:544-51.
Kohno M, Kawahito Y, Tsubouchi Y, Hashiramoto A, Yamada R, Inoue K, Kusaka Y, Kubo T, Elenkov E, Chrousos G P, Kondo M, Sano H. (2001). Urocortin expression in synovium of patients with rheumatoid arthritis and osteoarthritis: relation to inflammatory activity. J Clin Endocrinol Metab 86:4344-4352
Martinez V, Wang L, Rivier J, Vale W, Tache Y. (2002) Differential actions of peripheral corticotropin releasing factor and urocortin II and III on gastric emptying and colonic transit in mice: role of CRF receptor subtypes 1 and 2. J Pharm Exp Ther 301:611-617
Million M, Maillot C, Saunders P, Rivier J, Vale W, Tache Y. (2002). Human urocortin II, a new CRF-related peptide, displays selective CRF2-mediated action on gastric transit in rats. Am J Physiol 282:34-40
Muramatsu Y, Fukushima K, Iino K, Totsune K, Takahashi K, Suzuki T, Hirasawa G, Takeyama J, Ito M, Nose M, Tashiro A, Hongo M, Oki Y, Nagura H, Sasano H. (2000) Urocortin and corticotropin-releasing factor receptor expression in the human colonic mucosa. Peptides. 21:1799-1809.
Nishikimi T, Miyata A, Horio T, Yoshihara F, Nagaya N, Takishita S, Yutani C, Matsuo H, Matsuoka H, Kangawa K. (2000) Urocortin, a member of the corticotropin-releasing factor family, in normal and diseased heart. Am J Physiol 279:3031-3039
Pedersen W A, Wan R, Zhang P, Mattson M P. (2002) Urocortin, but not urocortin II, protects cultured hippocampal neurons from oxidative and excitotoxic cell death via corticotropin releasing hormone receptor type I. J Neurosci 22:404-412
Perrin, M. H., and W. W. Vale. (1999). Corticotropin releasing factor receptors and their ligand family. Ann N Y Acad Sci 885:312-28.
Poliak S, Mor F, Conlon P, Wong T, Ling N, Rivier J, Vale W, Steinman L. (1997) Stress and autoimmunity: the neuropeptides corticotropin-releasing factor and urocortin suppress encephalomyelitis via effects on both the hypothalamic-pituitary-adrenal axis and the immune system. J Immunol. 158:5751-5756.
Theoharides T C, Singh L K, Boucher W, Pang X, Letourneau R, Webster E, Chrousos G. (1998). Corticotropin-releasing hormone induces skin mast cell degranulation and increased vascular permeability, a possible explanation for its proinflammatory effects Endocrinology 139:403-408
Tsatsanis C, Androulidaki A, Dermitzaki E, Gravanis A, Margioris A N. Differential effects of corticotropin-releasing hormone (CRH) and its related pepdide urocortin and urocortin II on macrophage apoptosis (submitted)
Turnbull A V, Vale W, Rivier C. (1996) Urocortin, a corticotropin-releasing factor-related mammalian peptide, inhibits edema due to thermal injury in rats. Eur J Pharmacol. 303:213-216.

Vaughan J, Donaldson C, Bittencourt J, Perrin M H, Lewis K, Sutton S, Chan R, Turnbull A V, Lovejoy D, Rivier C. (1995) Urocortin, a mammalian neuropeptide related to fish urotensin I and to corticotropin-releasing factor. Nature 778:223-234
Webster, E. L., D. E. Tracey, M. A. Jutila, S. A. Wolfe, Jr., and E. B. De Souza. (1990). Corticotropin-releasing factor receptors in mouse spleen: identification of receptor-bearing cells as resident macrophages. Endocrinology 127:440-52.
Webster, E. L., D. J. Torpy, I. J. Elenkov, and G. P. Chrousos. (1998). Corticotropin-releasing hormone and inflammation. Ann N Y Acad Sci 840:21-32.

1. A method for the treatment of an inflammatory disease or condition comprising administering a pharmaceutical composition comprising one or more synthetic corticotrophin-releasing hormone receptor 1 (“CRH-R1”) antagonists and/or corticotrophin-releasing hormone receptor 2 (“CHR-R2”) agonists to a patient in need thereof, wherein the one or more synthetic CRH-R1 antagonists and/or CHR-R2 agonists is directly modifying the response of monocyte/macrophage cell activation, proliferation, differentiation or apoptosis.

2. The method according to claim 1, wherein the composition is formulated for local or systemic administration.

3. The method according to claim 1, wherein the composition further comprises usual exhibients such as diluents, fillers, binders, disintegrants, lubricants, conserving agents, flavourings and colourings.

4. The method according to claim 1, wherein the formulation is formulated for oral, parenteral or intradermal administration.

5. The method according to claim 4, wherein the composition is formulated as an injection liquid.

6. The method according to claim 1, wherein the one or more synthetic CRH-R1 antagonist and/or CRHR2 agonist comprises antalarmin.

7. The method according to claim 6, wherein the one of more synthetic CRH-R1 antagonist and/or CRH-R2 agonist is antalarmin.
